(19) United States
(12) Patent Application Publication
(10) Pub. No.: US 2010/0061889 A1
(43) Pub. Date: Mar. 11, 2010

(54) APPARATUS AND METHOD FOR PROVIDING RESULT OR URINE AND/OR GAS ANALYSIS

(76) Inventor: Yi-Seul Park, Seoul (KR)

Correspondence Address: SHERR & VAUGHN, PLLC, 620 HERNDON PARKWAY, SUITE 320, HERNDON, VA, 20170, US

(21) Appl. No.: 12/522,151

(22) PCT Filed: Feb. 21, 2007
(86) PCT No.: PCT/KR07/00911

§ 371 Date: Jul. 20, 2009

(30) Foreign Application Priority Data
Jan. 2, 2007 (KR) 10-2007-0000043

(51) Int. Cl. G01N 33/48

(52) U.S. Cl. 422/55; 422/68.1

(57) ABSTRACT

The present invention relates to a device for providing a result of urine and feces gas analysis result by analyzing the urine and/or feces gas flowing into a toilet so as to acquire status in formation, and displaying advertisements corresponding to the acquired information. According to the present invention, while a user is performing excretion, the user may obtain information associated with his or her health based upon an analysis of his or her urine and/or feces, and an advertiser may provide consumers with advertisements associated with the user's health.

### TECHNICAL FIELD

The present invention relates to an apparatus and method for providing result of urine and/or gas analysis. Most particularly, the present invention relates to an apparatus and method for analyzing urine and/or gas flowing into a urinal or toilet and displaying the analyzed result.

### BACKGROUND ART

Human excrement, such as urine and feces, and blood are one of the most important bases indicating an individual's present health conditions and status. Being expelled from the human body through a natural and routine metabolic process, urine or feces may be easily collected, as opposed to blood. Also, by analyzing the collected urine or gas, a variety of information related to the individual's present health condition and status may be retrieved.

Generally, in order to verify the condition of one's urine without having to visit a clinical facility, one contacts his or her urine on a stick or piece of paper containing a testing agent, thereby observing the color changes in the testing stick or paper. Alternatively, one may use a compact mobile meter that is used for checking the water purity level of an aquarium or swimming pool so as to verify the level of hydrogen ion concentration.

However, the above-described methods are disadvantageous in that the user is required to purchase or carry the devices required for testing his or her urine, which leads to an inconvenience, and also in that each test may be performed one time only. Furthermore, the general public who lack medical knowledge may not be able to make an accurate judgment of the test results. Therefore, the user may not be able to take the proper and necessary measures in accordance with the test results.

### DISCLOSURE OF INVENTION

### Technical Problem

An object of the present invention devised to solve the problem lies on providing an apparatus and method for providing result of urine and/or gas analysis that can perform simple real-time tests of a user's urine and gas through a metabolic process, which is referred to as excretion, without having to waste surplus money, time, and effort on a separate test, thereby providing information with respect to the test results.

The object of the present invention can be achieved by providing, in an apparatus for providing result of urine analysis, the apparatus including a sensor unit fixed inside a toilet, so as to come in contact with urine flown into the toilet, thereby acquiring numerical level information from the inflowing urine, a processing unit analyzing the urine by using the numerical level information acquired from the sensor unit, thereby acquiring real-time urine status information and generating output information based upon the urine status information, and a display unit fixed in a position visible and readable by a user of the toilet and displaying the output information inputted from the processing unit.

In another aspect of the present invention, provided herein, in an apparatus for providing result of feces gas analysis, the apparatus includes a sensor unit fixed inside a toilet, so as to come in contact with feces gas flown into the toilet, thereby acquiring element information from the inflowing feces gas, a processing unit acquiring feces gas status information based upon the element information acquired from the sensor unit, thereby generating output information based upon the feces gas status information, and a display unit fixed in a position visible and readable by a user of the toilet and displaying the output information inputted from the processing unit.

In another aspect of the present invention, provided herein, in a system for providing result of urine analysis, the system includes an advertisement providing server for registering an advertisement based upon the result of urine analysis, and a device for providing result of urine analysis storing an advertisement downloaded from the advertisement providing server, and searching and displaying an advertisement corresponding to the result of urine analysis of a user from the downloaded advertisement.

In a further aspect of the present invention, provided herein, in a method for providing advertisement using a result of urine analysis, the method includes the steps of downloading and storing an advertisement from an advertisement providing server, analyzing urine of a user, thereby acquiring a result information, searching an advertisement corresponding to the acquired result information from the downloaded advertisement, and displaying the searched advertisement.

Technical Solution

In the present invention, test results of urine and/or gas analysis may be provided at the same time the user produces excrement without having to purchase a separate device for testing his or her urine and/or gas or having to visit a hospital. Also, by providing an advertisement based upon the real-time analysis result of the user's urine and/or feces, the user may be able to approach products necessary to maintain one's health more easily. Accordingly, an advertiser may also be able to provide an effective advertisement to the public. Furthermore, a system provider may be able to charge fees for each advertisement.

### BRIEF DESCRIPTION OF THE DRAWINGS

The accompanying drawings, which are included to provide a further understanding of the invention, illustrate embodiments of the invention and together with the description serve to explain the principle of the invention.

In the drawings:

FIG. 1 illustrates a block diagram showing a structure of a system for providing result of urine and/or gas analysis according to an embodiment of the present invention.

FIG. 2 illustrates a block diagram showing a processing unit of the system for providing result of urine and/or gas analysis according to the present invention.

FIG. 3 illustrates a block diagram showing a system for providing result of urine and/or gas analysis according to the present invention, the system having an advertisement management function.

FIG. 4 illustrates a block diagram showing a processing unit of the system for providing result of urine and/or gas analysis according to the present invention, the processing unit having an advertisement management function.

FIG. 5 illustrates a flow chart showing a method for providing result of urine and/or gas analysis according to an embodiment of the present invention.

FIG. 6 illustrates a structure of a flush urinal for male usage according to the present invention.

FIG. 7 illustrates a structure of a flush toilet according to an embodiment of the present invention.

FIG. 8 illustrates a structure of a flush toilet according to another embodiment of the present invention.

FIG. 9 illustrates a structure of a vacuum toilet according to an embodiment of the present invention.

### BEST MODE FOR CARRYING OUT THE INVENTION

The present invention uses urine, which is one of the most variable health condition indexes within the human body, and which is also one of the most easily collectable elements of the human body, thereby providing a user with information based upon results urine and/or gas analysis of the user through a display screen or speaker located at n eye level of the user, while the user is performing excretion. In addition, as information based upon the result of the urine analysis, the present invention outputs an advertisement corresponding to the urine analysis result.

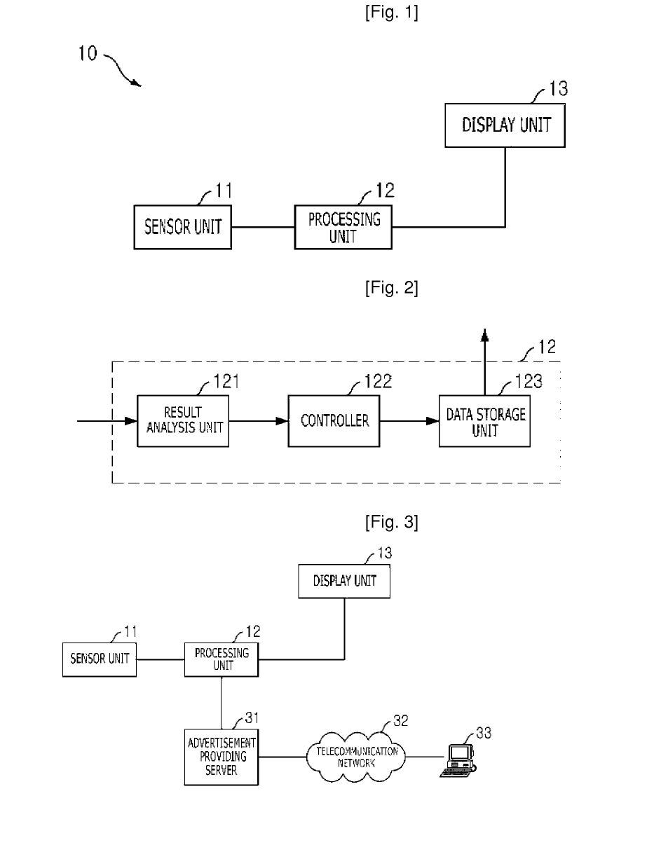

FIG. 1 illustrates a block diagram showing a structure of a system for providing result of urine and/or gas analysis according to an embodiment of the present invention. Referring to FIG. 1, the system for providing result of urine and/or gas analysis 10 includes a sensor unit 11 for directly contacting with urine for urine analysis or for contacting gas, thereby acquiring numerical value information corresponding to urine and/or feces elements or gas element information, a processing unit 12 for reading and outputting analysis result by using the numerical value information acquired by the sensor unit 11, and a display unit 13 for outputting an output signal of the processing unit through an image and/or sound.

The sensor unit 11 is provided with a sensor for acquiring the numerical value information of the urine, and a sensor for acquiring gas information of the feces. In the sensor unit 11, the sensor for acquiring the numerical value information of the urine may be fixed in a position inside of a toilet or urinal, which comes in direct contact with the urine, or fixed in a position where urine forms a pool. Meanwhile, it is preferable for the sensor for acquiring gas information of the feces to be fixed in a position inside of a toilet, which may easily come in contact with gas. An example of the acquired numerical value information of urine includes hydrogen ion concentration (pH) and electric conductivity of the urine. Meanwhile, an example of the acquired gas information includes gas element information acquired by analyzing gas odor. The numerical value information of urine and/or gas information is transmitted from the sensor unit 11 to the processing unit 12 through a wireless or wired network.

FIG. 2 illustrates a block diagram showing a processing unit of the system for providing result of urine and/or gas analysis according to the present invention. Referring to FIG. 2, the numerical value information of urine and/or gas information acquired from the sensor unit 12 is transmitted to a result analysis unit 121. The result analysis unit uses the received information to read and output analysis result of the urine test and/or gas test.

For example, by using a pH of the urine, the user can verify whether he or she is consuming adequate amount of essential minerals. More specifically, urine of a normal status is subacidic. However, if the urine status exceeds the normal acidic level, then the user may determine that he or she is consuming food with high content in oil or fat or consuming a large amount of junk food. Therefore, the present invention may display advertisements advising the user to consume organic vegetables. Meanwhile, the user may also verify whether he or she is consuming adequate amount of water by using the electric conductivity of his or her urine. If the conductivity level is higher than the normal level, then the present invention may display an advertisement proposing the user to consume a larger quantity of water.

The above-described advertisement messages and/or voice information are stored in a data storage unit 123. Therefore, a controller 122 searches for the proper text message and/or voice information corresponding to the urine and/or gas analysis result from the data storage unit 123, thereby inputting the searched output information to the display unit 13.

During searching process, the data storage unit 123 may not only store the information acquired from urine and/or feces but may also store data considering external environment status information. Therefore, the controller 122 may not only search the information acquired from urine and/or feces but may also search output data considering external environment status information. Examples of the external environment status information include temperature, humidity, time, and season. For example, if the urine is of a dark color and forms a considerable amount of foam during the summer season, the user may be dehydrated due to the hot weather. Therefore, the controller 122 may search the data storage unit 123 for a text message such as “The weather is very hot. Drink 2 bottles of XX brand ionized beverage to replenish your bodily water level.”

Meanwhile, the inputted output information is displayed on the display unit 13. Examples of the output information include advertisements through text message, voice, and visual data.

FIG. 3 illustrates a block diagram showing a system for providing result of urine and/or gas analysis according to the present invention, the system having an advertisement management function. As shown in FIG. 3, a company requesting advertisement 33 registers an advertisement corresponding to the numerical level information of urine to the advertisement providing server 31. At this point, the company requesting advertisement 33 may be charged with a predetermined fee depending upon the type of the advertisement or depending upon the amount of information including text and/voice for configuring the particular advertisement. Meanwhile, a predetermined fee may also be charged depending upon the number of times the advertisement is being displayed.

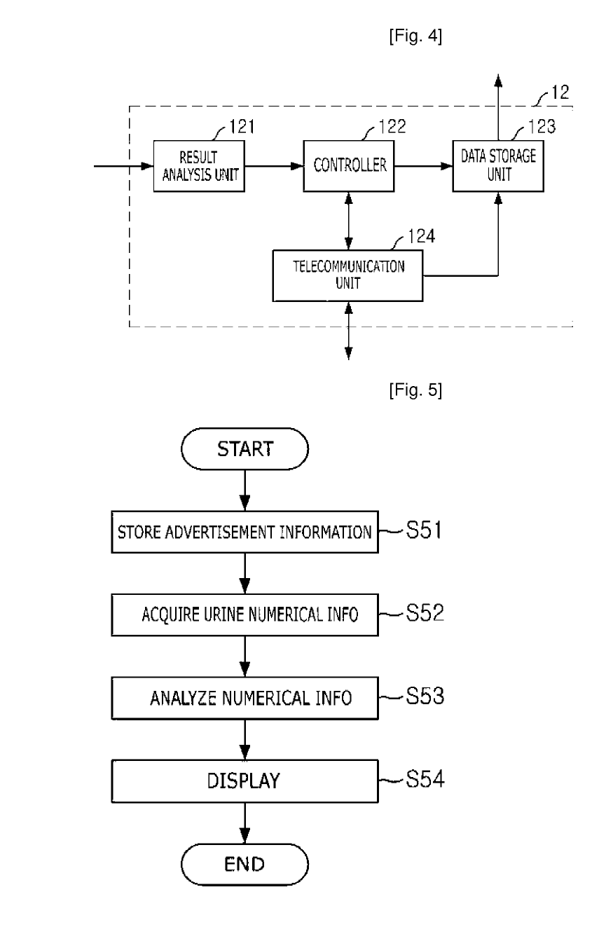

FIG. 4 illustrates a block diagram showing a processing unit of the system for providing result of urine and/or gas analysis according to the present invention, the processing unit having an advertisement management function. Referring to FIG. 4, the processing unit includes a result analysis unit 121, a controller 122, a data storage unit 123, and a communication unit 124 for performing communication with the advertisement providing server 31. Herein, the communication between the processing unit and the advertisement providing server is performed via a wireless or wired network.

Depending upon the control of the controller 122, the communication unit 124 stores the contents of the urine information and/or gas information received from the advertisement providing server 31 into the data storage unit 123. Accordingly, the controller 122 searches the contents of the advertisement corresponding to the analysis result, and controls the display unit 13 so that the searched advertisement is inputted to the display unit 13.

Meanwhile, the controller 122 may calculate how frequently a particular advertisement has been displayed. Also, depending upon the number of advertisements and/or the amount of data, a fee may be charged to the advertisement company.

FIG. 5 illustrates a flow chart showing a method for providing result of urine and/or gas analysis according to an embodiment of the present invention. Referring to FIG. 5, the system for providing result of urine and/or gas analysis receives advertisement information, which corresponds to the analysis information of urine and/or feces, through a wired/wireless network and stores the received advertisement information (S51). Meanwhile, the advertisement information may be inputted, without passing through the wired/wireless network, by directly connecting an inputting/outputting device to the processing unit of the method for providing result of urine and/or gas analysis.

The sensor unit of the system for providing result of urine and/or gas analysis acquires information on urine and/or feces (S52). And, the result analysis unit analyzes the result based upon information on the urine and/or gas analysis (S53). The controller of the system for providing result of urine analysis uses the analysis result so that the information corresponding to the analysis result is outputted to the display unit (S54). Furthermore, the controller may calculate a number of advertisements and/or the amount of data so as to charge a fee to the advertiser.

A method for fixing a sensor unit in accordance with the structure of each toilet type will now be described.

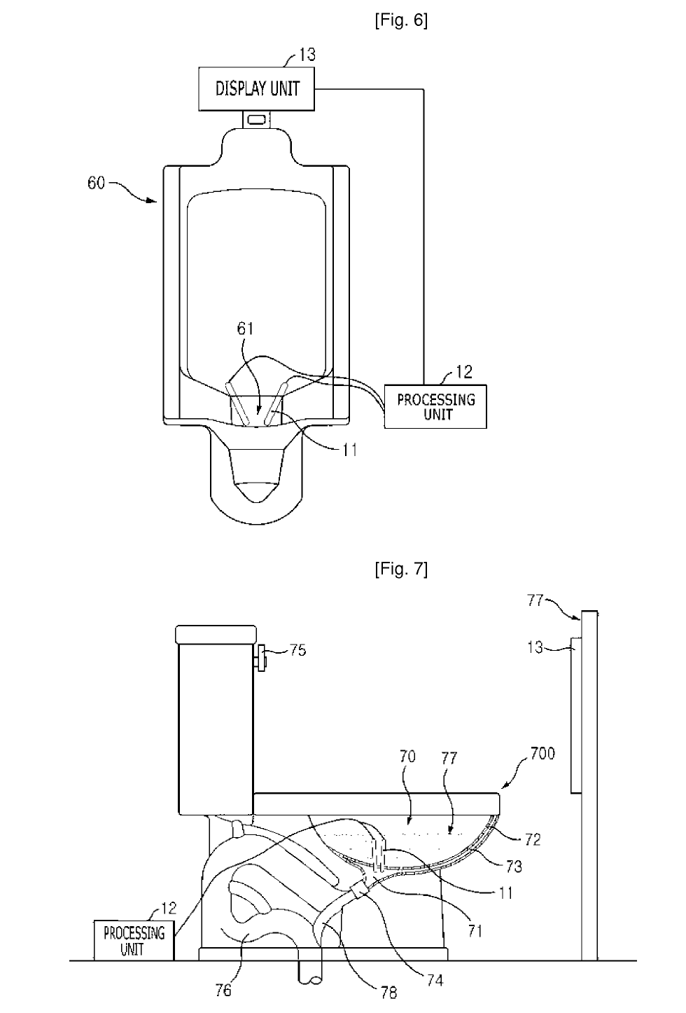

FIG. 6 illustrates a structure of a flush urinal for male usage according to the present invention. Referring to FIG. 6, when using the flush urinal 60 for male usage, the sensor unit 11 is provided in a urine collector 61, wherein the urine forms a pool before the user drains water to wash away the urine. More specifically, the sensor unit 11 is fixed to come into direct contact with the pool of urine in the urine collector 61.

When urine is flown into the flush urinal 60 for male usage according to the present invention, the inflown urine forms a pool in the urine collector 61. Thus, the sensor unit 11 acquires numerical level information on urine from the pool of urine. Then, the result of the acquired information is transmitted to the processing unit 12, and a text and/or voice message corresponding to the analysis result of the processing unit 12 is displayed on the displaying unit 13. Herein, the transmission of information from the sensor unit 11 to the processing unit 12 and the transmission of information from the processing unit 12 to the display unit 13 may be performed in both wired and wireless methods.

Referring to FIG. 6, when using the flush urinal for male usage, it is preferable that the display unit 13 is fixed at the eye level of the user. The display unit 13 includes a text and/or voice message, such as information associated to the user's health, corresponding to the analysis result of the urine currently being flown in. For example, information such as “You have not consumed enough water. Please consume 2 glasses of water.” may be displayed. For example, an advertisement such as “You have not consumed enough vegetables. Please consume 00 brand fresh organic vegetables. corresponding to the analysis result of the user's urine may be displayed. Accordingly, the health information is provided to the user, and the advertiser may profit from the advertisement effect.

In the flush urinal for male usage, when the user finishes urination, water is flushed in either automatically or by manual operation of the user so as to wash out the urine within the urinal. Accordingly, when water is flown in to wash out the urine, the controller detects this process through a water inflow sensor, thereby initializing the screen that is used for displaying messages. Then, the urinal stands-by at an initialized status for the next user. Meanwhile, if water is flown in by the manual operation of the user, when the user operates the water inflow button or lever, the display screen is initialized, thereby waiting for the next user in a stand-by mode. The initialized display screen may display a message such as Please check your health condition through your own urine.

FIG. 7 illustrates a structure of a flush toilet according to an embodiment of the present invention. Referring to FIG. 7, when a flush toilet 700 according to an embodiment of the present invention is used, unlike the general flush toilet, the flush toilet 700 is provided with a urine collector 71, wherein a pool of urine may be formed, at a lower area of a feces chamber 70 of the flush toilet. It is preferable to adopt this embodiment of the present invention when using a stick-type sensor in the sensor unit.

In case of the conventional flush toilet, when performing excretion, urine and feces are both collected in the same feces chamber. Therefore, it is difficult to acquire urine information from the water pooled in the feces chamber of the flush toilet.

However, since the contact point of the urine and the contact point of the feces are different from one another when the user is performing excretion, in order to perform an instantaneous collection of urine while the user is urinating, a urine collector 71 for collecting urine is placed in an area within the flush toilet, so as to be differentiated from the feces chamber. Therefore, since the urine expelled from the user's body is not mixed with the water pooled in the flush toilet, accurate urine information may be acquired.

The structure of the flush toilet having the urine collector 71 fixed therein so as to collect inflowing urine will now be described in detail. As shown in FIG. 7, a urine collecting hole 72 is fixed at a position of the flush toilet whereby urine is directly contacted. Therefore, when urine is flown in, the urine is flown into the urine collecting hole 72. At this point, it is preferable that the urine collecting hole is fixed at a position higher than the usual water level 77 within the toilet.

When urine is flown inside the urine collecting hole 72, the urine flows through a urine collecting guide 73 so as to form a pool in the urine collector 71. Then, since a sensor unit 11 is fixed in the urine collector 71, the urine numerical level information may be accurately acquired. The sensor 11 is fixed on a side surface of the space configuring the urine collector 71, and the space of the urine collector 71 or the space within the urine collector 71 wherein the sensor unit 11 is fixed may be modified into various shapes depending upon the shape and size of the sensor unit 11. For example, when using the stick-type sensor, since the sensor is designed to have a portion of its body dipped into a solution (i.e. , urine) to be measured, thereby measuring the numerical level of the urine, a urine collector 71 having the shape of a cavity hole is formed so that urine pooled in the cavity hole can be used to be measured.

Meanwhile, it is preferable to fix an electronic nose for acquiring gas information from the feces above the water surface within the feces chamber of the toilet.

When the urine within the urine collector 71 is expelled, the urine is either directly expelled through a drain pipe 76, through which feces and water within the feces chamber 70 are expelled, or expelled through a urine discharge pipe 78 connected to the drain pipe 76. At this point, the lower end of the urine collector 71 is designed to be opened and closed by a solenoid valve 74. The opening and closing of the solenoid valve 74 may be controlled and operated in accordance with a water inflow sensor. More specifically, when water is flown into the feces chamber (an urine collector) in order to wash the inside of the toilet after performing excretion, the solenoid valve 74 at the lower end of the urine collector 71 is opened so that the urine pooled in the urine collector 71 is expelled and discharged to the drain pipe 76. Additionally, the opening and closing of the solenoid valve 74 may be controlled by a washing button (or lever). More specifically, when a flush button (or lever) 75 is pushed in order to wash the inside of the toilet after performing excretion, a flush button (or lever) sensor detects the pushing of the button (or lever), thereby opening the solenoid valve 74 so that the urine collected in the urine collector 71 may be discharged to the drain pipe 76. After discharging the urine within the urine within the urine collector 71, it is preferable to close the solenoid valve 74 so as to prevent unpleasant odor from flowing in through the drain pipe 76.

As shown in FIG. 7, in using a flush toilet, it is preferable that the display unit 13 is fixed on an inner surface of the bathroom door or on a surface of the wall 77 to which the user faces during excretion.

In the flush toilet 700, when the user finishes urination, water is flushed in either automatically or by manual operation of the user so as to wash out the urine and/or feces within the toilet. Accordingly, when water is flown in to wash out the urine, the controller detects this process through a water inflow sensor, thereby initializing the screen that is used for displaying messages. Then, the urinal stands-by at an initialized status for the next user. Meanwhile, if water is flown in by the manual operation of the user, when the user operates the water inflow button or lever without being provided with a water inflow sensor, the display screen is initialized, thereby waiting for the next user in a stand-by mode. The initialized display screen may display a message such as Please check your health condition through your own urine.

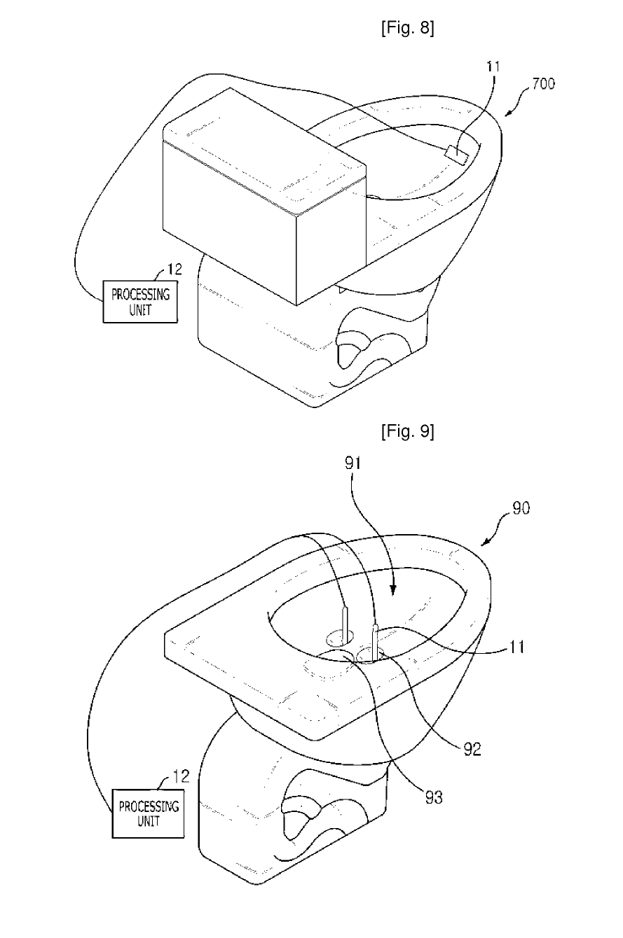

FIG. 8 illustrates a structure of a flush toilet according to another embodiment of the present invention. According to this embodiment, by allowing the sensor unit 11 to be directly exposed to the inflowing urine, urine numerical level information may be acquired before the urine is mixed with water within the feces chamber, even without having to fix a separate urine chamber within the flush toilet. In this embodiment, it is preferable to use a panel-type sensor as the sensor unit 11, which can be adhered on the surface of the urine chamber, instead of the stick-type sensor.

FIG. 9 illustrates a structure of a vacuum toilet according to an embodiment of the present invention. As shown in FIG. 9, when using the vacuum toilet 90, a urine collector 92 is provided on an inner surface of a toilet trap 91 so as to allow the urine of the user to be pooled (or collected) therein.

In case of the vacuum toilet, although water is not provided within the toilet, urine and feces are simultaneously collected within the toilet trap 91 during excretion. Therefore, in order to separately acquire urine numerical level information from the urine without being mixed with the feces, a urine collector 92 is fixed at a predetermined portion within the toilet. It is preferable to form the urine collected in the form of a cavity hole so that even the smallest amount of urine may be collected therein. The urine collector 92 includes a sensor unit 11 that can acquire urine numerical level information from the urine collected within the urine collector 92.

Meanwhile, as shown in the embodiment of the flush toilet, a urine collecting hole is fixed at a position of the vacuum toilet whereby urine is directly contacted. Therefore, when urine is flown in, the urine is flown into the urine collecting hole, and the urine flown through the urine collecting hole is collected in a urine chamber through a urine collecting guide, so as to acquire urine numerical level information from the collected urine. As described above, when acquiring urine numerical level information from the collected urine, it is preferable to include a solenoid valve which is opened and closed in order to discharge the urine collected in the urine collector.

Also, by allowing the sensor unit 11 to be directly exposed to the inflowing urine, urine numerical level information may be acquired before the urine is mixed with water within the feces chamber, even without having to fix a separate urine chamber within the vacuum toilet. In this embodiment, it is preferable to use a panel-type sensor as the sensor unit 11, which can be adhered on the surface of the urine chamber, instead of the stick-type sensor.

It is preferable to fix an electronic nose for contacting gas so as to acquire gas information from the feces on an inner surface of the toilet trap within the toilet.

In using the vacuum toilet 90, when the user finishes excretion, an opening part 93 is automatically or manually opened by a manual operation of the user. Thus, water is flown in, thereby discharging the urine and feces through a vacuum pipe.

Accordingly, when water is flown in to wash out the urine, the controller detects this process through a water inflow sensor, thereby initializing the screen that is used for displaying messages. Then, the urinal stands-by at an initialized status for the next user. Meanwhile, if water is flown in by the manual operation of the user, when the user operates the water inflow button or lever without being provided with a water inflow sensor, the display screen is initialized, thereby waiting for the next user in a stand-by mode. The initialized display screen may display a message such as “Please check your health condition through your own urine.”

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### INDUSTRIAL APPLICABILITY

The present invention may be applied as a medical device for users and may also be applied in advertisement industries for a system provider.

1-33. (canceled)

34. An apparatus for providing results of urine analysis, the apparatus comprising:
a sensor unit fixed inside a toilet, configured to come in contact with urine which has flowed into the toilet, thereby acquiring numerical level information from the inflowing urine;
a processing unit configured to analyze the urine by using the numerical level information acquired from the sensor unit, thereby acquiring real-time urine status information and generating output information based upon the urine status information; and
a display unit fixed in a position visible and readable by a user of the toilet and
displaying the output information inputted from the processing unit.

35. The apparatus of claim 34, wherein the urine numerical level information includes at least one of hydrogen ion concentration (pH) and electric conductivity of the urine.

36. The apparatus of claim 35, wherein the output information based upon the status information is an advertisement corresponding to the status information.

37. The apparatus of claim 36, wherein the toilet is a flush urinal for male usage, and wherein the sensor unit is fixed to a urine collector in which the inflowing urine is collected.

38. The apparatus of claim 37, wherein the processing unit is configured to use one of wireless telecommunication and wired telecommunication so as to receive the urine numerical level information acquired from the sensor unit.

39. The apparatus of claim 37, wherein the display unit is fixed at a position corresponding to an eye level of the user above the flush urinal for male usage.

40. The apparatus of claim 36, wherein the toilet is a flush toilet.

41. The apparatus of claim 40, wherein the flush toilet comprises:
a feces chamber formed with a urine collecting hole, the urine collecting hole
allowing inflowing urine to flow therein;
a urine guide, formed on an inner wall inside the feces chamber, and shaped to guide the inflowing urine into the urine collecting hole; and
a urine collector to collect the inflowing urine,
wherein the sensor unit is fixed on the urine collector.

42. The apparatus of claim 41, further comprising a solenoid valve configured to open and close so as to discharge the collected urine to a lower portion of the urine collector.

43. The apparatus of claim 41, wherein the processing unit uses one of wireless telecommunication and wired telecommunication so as to receive the urine numerical level information acquired from the sensor unit.

44. The apparatus of claim 40, wherein the sensor unit is fixed on an inner wall of the flush toilet so as to come in direct contact with the inflowing urine.

45. The apparatus of claim 40, wherein the display unit is fixed on a wall surface facing the flush toilet in a lavatory.

46. The apparatus of claim 40, wherein the display unit is fixed on a door surface facing the flush toilet in a lavatory.

47. The apparatus of claim 36, wherein the toilet is a vacuum toilet, and wherein the sensor unit is fixed inside a trap having the urine and feces deposited therein and collected.

48. The apparatus of claim 47, wherein the trap of the vacuum toilet forms at least one cavity hole so as to have the inflowing urine collected therein, and wherein the sensor unit is fixed in the cavity hole.

49. The apparatus of claim 48, wherein the processing unit is configured to use one of wireless telecommunication and wired telecommunication so as to receive the urine numerical level information acquired from the sensor unit.

50. The apparatus of claim 47, wherein the display unit is fixed on a wall surface facing the vacuum toilet in a lavatory.

51. The apparatus of claim 47, wherein the display unit is fixed on a door surface facing the vacuum toilet in a lavatory.

52. The apparatus of claim 36, wherein the processing unit comprises:
a result analysis unit configured to use the numerical level information acquired from the sensor unit to analyze the urine;
a data storage unit configured to store advertisement data corresponding to the analysis result; and
a controller configured to control the processing unit so that the advertisement corresponding to the analysis result acquired from the result analysis unit is transmitted from the data storage unit to the display unit.

53. The apparatus of claim 52, wherein the processing unit further comprises a telecommunication unit configured to receive advertisement information transmitted from an external device based upon the control of the controller.

54. The apparatus of claim 53, wherein advertisement information received from the telecommunication unit is stored in the data storage unit, based upon the control of the controller.

55. The apparatus of claim 52, wherein the controller is configured to calculate a frequency level of each advertisement being displayed.

56. A system for providing result of urine analysis, the system comprising:
an advertisement providing server for registering an advertisement based upon the result of urine analysis; and
a device for providing results of urine analysis, storing an advertisement downloaded from the advertisement providing server, and searching and displaying an advertisement corresponding to the result of urine analysis of a user from the downloaded advertisement.

57. The system of claim 56, wherein the advertisement providing server includes charging fee information based upon a number of times each advertisement is being displayed.

58. The system of claim 56, wherein the advertisement being displayed comprises voice, text, and visual data.

59. A method for providing advertisement using a result of urine analysis, the method comprising the steps of:
downloading and storing an advertisement from an advertisement providing server;
analyzing urine of a user, thereby acquiring a result information;
searching an advertisement corresponding to the acquired result information from the downloaded advertisement; and
displaying the searched advertisement.

60. The method of claim 59, further comprising a step of connecting to the advertisement providing server through a telecommunication network and registering the advertisement.

61. The method of claim 60, further comprising the steps of:
checking a number of times each advertisement is being displayed; and
charging fees based upon a number of times each advertisement is being displayed.

62. An apparatus for providing result of feces gas analysis, the apparatus comprising:
a sensor unit fixed inside a toilet, configured to come in contact with feces gas which has flowed into the toilet, thereby acquiring element information from the feces gas;
a processing unit configured to acquire feces gas status information based upon element information acquired from the sensor unit, thereby generating output information based upon the feces gas status information; and
a display unit fixed in a position visible and readable by a user of the toilet and displaying the output information inputted from the processing unit.

63. The device of claim 62, wherein the processing unit comprises:
a result analysis unit configured to use the feces gas element information acquired from the sensor unit to analyze the feces gas;
a data storage unit configured to store advertisement data corresponding to the analysis result; and
a controller configured to control the processing unit so that the advertisement corresponding to the analysis result acquired from the result analysis unit is transmitted from the data storage unit to the display unit.

64. The apparatus of claim 63, wherein the processing unit further comprises a telecommunication unit configured to receive advertisement information transmitted from an external device based upon the control of the controller.

65. The apparatus of claim 64, wherein advertisement information received from the telecommunication unit is stored in the data storage unit, based upon the control of the controller.

66. The apparatus of claim 63, wherein the sensor unit corresponds to an electronic nose.
